(19) United States
(12) Patent Application Publication
(10) Pub. No.: US 2006/0096159 A1
(43) Pub. Date: May 11, 2006

(54) Method for improving the long term stability of biodiesel

(76) Inventors: Rudolf Bonsch, Mainz-Laubenheim (DE); Wolfgang Kastl, Offenbach am Main (DE); Pete Mitschke, Maintal (DE); Helmut Saft, Niedertal (DE)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/537,725

(22) PCT Filed: Sep. 23, 2003
(86) PCT No.: PCT/EP03/10550

§ 371 Date: Nov. 8, 2005

(30) Foreign Application Priority Data
Dec. 7, 2002 (DE) 102 57 215.1

(51) Int. Cl. C10L 1/18; C07C 51/43

(52) U.S. Cl. 44/385; 554/174

(57) ABSTRACT

A method for improvement of the long term stability of biodiesel is described, in which the crude ester produced by transesterification of a vegetable or animal fat or oil with methanol, with a strong acid or with a mixture of a strong acid and a complex former is subsequently treated intensively and the ester layer separated from the emulsion formed thereby is subjected to a thorough water washing and subsequently dried.

The object of the invention is a method for the manufacture of improved biodiesel from fats and oils, especially from oil seeds, from which the oil is obtained by pressing or by solvent extraction, as well as from Yellow Grease.

Biodiesel is known to be produced by transesterification of untreated glycerin esters, in Europe above all from rapeseed oil, by use of methanol and the addition either of acids (p-toluenesulfonic acid) or basic catalysts, such as for example KOH, NaOH or sodium methylate. In that connection batch processes as well as continuous processes are utilized.

The vegetable and animal crude oils employed for the production of biodiesel always contain a proportion of free fatty acids and mucilages, which in the case of base-catalyzed transesterification lead to a higher catalyst consumption by saponification of the free fatty acids which are then removed by way of the glycerin phase. The mucilages in the crude oil are converted by direct transesterification into stable emulsions, which have a negative effect on the separation of ester and glycerin phases. Therefore preferably de-acidified and de-mucilated oils with a free fatty acids content of <0.1% and a phosphorus content of <20 ppm are utilized for the production of biodiesel. An especially suitable method for the conversion of a vegetable or animal oil or fat with methanol or ethanol and an alkaline catalyst is described in German Patent Application 41 23 928, in which one operates with at least two conversion stages, wherein each conversion stage contains a mixed reactor and a separator for separation of a light ester-rich phase from a heavy glycerin-rich phase.

After the transesterification has taken place, the residual glyceride and the dissolved glycerin as well as the salts and the saponified fatty acids are extracted from the crude methyl ester. For this purpose the crude methyl ester is subjected to an acid treatment and subsequent water wash by use of centrifuging, stirrer vessels or water columns operating on the counter current principle. The fatty acid methyl ester produced thereby normally corresponds to the requirements of standard biodiesel.

Though it has been shown that storage of the pure biodiesel after washing and drying fulfills the analytical criteria of standard biodiesel, dependent on the fat or oil employed subsequent flocculation can occur, independently of whether the biodiesel is additionally filtered or centrifuged after drying. 97% of these flocculations consist of organic material with an ash content of 3%, which is made up of single or multivalent cations, sulfur and phosphorus compounds. These flocculations can deeply impair the usefulness of biodiesel as a fuel, because they lead rapidly to plugging of the fuel filter. It is therefore a critical prerequisite for the technical utility of biodiesel that also for longer storage the occurrence of flocculations be prevented with certainty.

In as much as it has been found that the occurrence of flocculations is based on condensation- or crystallization-nuclei such as phosphatides and ionic complexes of organic or inorganic nature, which are always contained in the conventionally manufactured biodiesel, the object is set of markedly lowering the quantity of crystallization nuclei by washing of the crude methyl ester so that subsequent flocculation is no longer observed in the dried ester, on storage.

The invention relates therefore to a method for improvement of the long term stability of biodiesel, in which the crude methyl ester produced by transesterification of a vegetable or animal fat or oil with methanol, is intensively post-treated with a strong acid or with a mixture of a strong acid and a complex former, and the ester layer separated from the emulsion layer formed thereby is subjected to a thorough water washing and is subsequently dried.

Subsequent treatment of the biodiesel suitably involves the use of a mechanically intensive mixer at temperatures between 25 and 60° C.

In accordance with the invention, hydrochloric acid, sulfuric acid or phosphoric acid are employed above all as strong acids, and as complex former EDTA or citric acid.

The invention is based on the recognition that the flakes subsequently falling out of the pure biodiesel are organic in nature and consist of materials which are normally dissolved in the fatty acid methylester and therefore also in biodiesel. This can include salts of long chain fatty acids, waxes or residual mucilage material. Following subsequent transesterification these dissolved compounds are no longer separable mechanically by application of centrifuges and filters in the processing of biodiesel. Also dissolved organic compounds which are present in such small quantities as to be barely detectable analytically can not be transferred with certainty from the ester phase into the aqueous phase by washing of the crude methyl ester with centrifuges or wash columns. Only in the presence of a “crystallization nucleus” onto which the dissolved, and in the case of the ester finely divided, compounds can be taken up, it comes eventually to the point of visible flake formation.

The aim of the method in accordance with the invention for improvement of the long term stability of biodiesel can thus not be to remove the actual dissolved organic compounds taken up into the ester phase, but to eliminate the remnants with regard to “crystallization nuclei”. These can be single or multivalent cations, residual soaps from fatty acids having multivalent cations, complexes of inorganic or organic compounds or phosphatides, or mucilage.

The removal of crystallization nuclei is achieved on the one hand by a more intensive washing process and on the other hand by an additional hydrophilization of the crystallization nuclei in order to make them more easily separated from the organic ester phase in the subsequent water wash, also at the same time an intensified splitting of the long chain calcium and magnesium soaps as well as to bring about the definite removal of iron compounds.

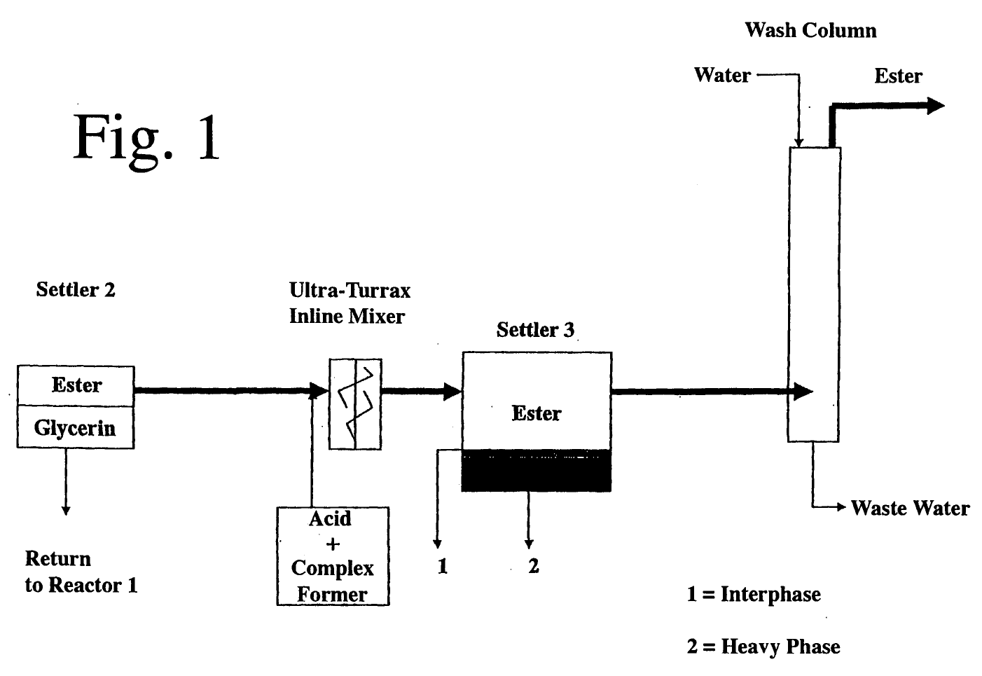

The method in accordance with the invention is explained in further detail by means of the appended FIG. 1.

The crude ester formed by transesterification of a vegetable or animal fat or oil or a Yellow Grease with methanol is initially present in a mixture with the split-off glycerin. This mixture contains as impurities, un-reacted methanol, soaps, free glycerin, mono and diglycerides, residual alkaline catalyst (for example sodium methylate) as well as phosphatide residues (mucilages) and organic and inorganic iron compounds from the fat or oil employed. In the Settler 2, the heavy phase containing the glycerin and residual catalyst is separated and fed back into reactor 1 employed for the transesterification.

The crude ester phase is on the other hand fed to an intensive mixing apparatus (Ultra-Turrax Inline Mixer) for splitting of the soaps with a strong acid or with a mixture of a strong acid and a complex former such as for example citric acid or EDTA. With an energy input of 0.002 kW/kg of crude ester, a fine emulsion is produced at about 50° C. in the special mixing chamber of the Inline Mixer from the ester and from the acid phase, which is fed to Settler 3.

After a waiting period of about 30 minutes a heavy phase separates from the ester and the emulsion breaks. A stable interphase develops between the ester phase and the heavy phase which is not separable into the heavy phase even with application of a commercial centrifuge.

The thus pre-purified ester is then fed to a subsequent water wash. This water wash may be carried out in a stirrer vessel or a centrifuge or in a wash column operated on the counter current principle. Quite especially an intensive washing with the Ultra Turrax Inline Mixer has proved effective.

The purified biodiesel thus produced is practically free of any condensation and crystallization nuclei as well as mucilage materials and iron compounds and has such outstanding long term stability that even after drying the biodiesel to remove residual traces of water, no cloudiness or flocculation occurs upon long term storage.

It is surprising moreover that with the method in accordance with the invention the utilization of an intensive mixer leads in the shortest time to splitting of the soaps by strong acids, and furthermore that in presence of strong acid, alcohol and water the phosphatides and iron compounds which are still present in the ester dissolve out of the organic phase and are carried over into the heavy aqueous phase and into the interphase. For the separation of these impurities, the combination of an intensive mixer with a downstream settler has proved to be excellent, whereas the phosphatide containing interphase layer was not definitively separated from the ester by utilization of a centrifuge.

By means of the method in accordance with the invention the long term stability of biodiesel was successfully increased considerably in an extremely simple way, so that cloudiness and flake formation can be entirely avoided, and thereby the up until now extremely damaging plugging of pores and filters can be forestalled in the utilization of biodiesel.

1. Method for improving the long term stability of biodiesel, wherein
1.1 crude methyl ester is formed by transesterification of a vegetable or animal fat or oil with methanol,
1.2 the crude methyl ester thus formed is intensively post-treated at temperatures between 25 and 60 C, with a strong acid or with a mixture of a strong acid and a complex former
characterized in that
1.3 a fine emulsion is prepared from the ester and the acid phase,
1.4 the ester layer separated from the emulsion formed thereby is subjected to a thorough water wash and subsequently dried.

2. The method according to claim 1, characterized in that, as strong acid hydrochloric acid, sulfuric acid, ptoluenesulfonic acid or phosphoric acid are employed and as complex former EDTA or citric acid.

3. The method according to claim 1, characterized in that the water wash of biodiesel is carried out in a wash column according to the counter current principle or by means of a mechanically intensive mixer.
